(19) United States
(12) Patent Application Publication
(10) Pub. No.: US 2008/0296401 A1
(43) Pub. Date: Dec. 4, 2008

(54) Fragrance Dispenser

(75) Inventors: James Edward Thompson, SR., Easton, MD (US); Ernest Edmond Dill, JR., Viola, DE (US); Conroy Richard Marth, Preston, MD (US)

Correspondence Address: KILPATRICK STOCKTON LLP, 1001 WEST FOURTH STREET, WINSTON-SALEM, NC, 27101, US

(73) Assignee: CELESTE INDUSTRIES CORPORATION, Easton, MD (US)

(21) Appl. No.: 11/756,286

(22) Filed: May 31, 2007

(51) Int. Cl. A61L 9/04

(52) U.S. Cl. 239/57

(57) ABSTRACT

A fragrance dispenser may have an inner surface and an outer surface defining a plurality of windows. The outer surface may be coupled to the inner surface such that a cavity is defined between the inner surface and the outer surface. The cavity may be configured to receive a fragrance strip, and wherein the fragrance dispenser is configured to allow a threaded portion of a bottle to extend through the fragrance dispenser and engage with a bottle base.

### FIELD OF THE INVENTION

The present invention relates to fragrance dispensers.

### BACKGROUND

Lavatories in public restrooms, particularly in confined spaces, such as in an airplane, may employ fragrance dispensers to reduce or eliminate odors. For example, a conventional fragrance dispenser may be affixed to a wall and may include a liquid that may be dispensed by aerosol spray into the lavatory. However, such dispensers may be easily tampered with by users of the lavatory. For example, the aerosol spray may be removed from a mounting bracket and dispensed in overwhelming quantities. Additional, conventional fragrance dispensers may require a separate mounting bracket to be installed in the lavatory.

Thus, it may be desirable to provide a fragrance dispenser that may be secured to an existing fixture within a lavatory, such as a soap dispenser, so that the fragrance dispenser may be easily installed, and may be relatively resistant to tampering.

### SUMMARY

Embodiments of the present invention provide fragrance dispensers. For example, one illustrative embodiment comprises an inner surface; and an outer surface defining at least one window. The outer surface is coupled to the inner surface such that a cavity is defined between the inner surface and the outer surface, and the cavity is configured to receive a fragrance strip. The fragrance dispenser is further configured to allow a threaded portion of a bottle to extend through the fragrance dispenser and engage with a bottle base.

This illustrative embodiment is mentioned not to limit or define the invention, but to provide an example to aid understanding thereof. Illustrative embodiments are discussed in the Detailed Description, and further description of the invention is provided there. Advantages offered by various embodiments of this invention may be further understood by examining this specification.

### BRIEF DESCRIPTION OF THE DRAWINGS

These and other features, aspects, and advantages of the present invention are better understood when the following Detailed Description is read with reference to the accompanying drawings, wherein:

FIG. 1 shows a soap bottle engaged with a fragrance dispenser according to one embodiment of the present invention;

FIG. 2 shows a fragrance dispenser according to one embodiment of the present invention;

FIGS. 3 and 4 show a first section of a fragrance dispenser according to one embodiment of the present invention;

FIG. 5 shows cross-sectional views of a first section of a fragrance dispenser according to one embodiment of the present invention;

FIGS. 6 and 7 show a second section of a fragrance dispenser according to one embodiment of the present invention;

FIG. 8 shows a half cross-section of a fragrance dispenser according one embodiment of the present invention;

FIGS. 9A-B show half cross-sections of fragrance dispensers according to two embodiments of the present invention;

FIG. 10 shows a cross section of a fragrance dispenser coupled to a base according to one embodiment of the present invention;

FIGS. 11A-C show a method of assembly of a fragrance dispenser according to one embodiment of the present invention; and

FIGS. 12A-B show a method of engaging a fragrance dispenser with a threaded portion of a bottle according to one embodiment of the present invention.

### DETAILED DESCRIPTION

In one illustrative embodiment of the present invention, a fragrance dispenser comprises two sections, a first section and a second section, configured to couple together to form a unitary structure. The unitary structure comprises a donut shape having a central opening that may allow a bottle, such as a soap dispenser bottle, to pass through the unitary structure. When the two sections are coupled, a cavity is formed between the two sections into which a fragrance strip may be disposed. In this illustrative embodiment, each of the first and second sections comprise a donut shape, and the second section is configured to fit within the first section. The first section defines the outer perimeter of the unitary structure, and comprises a plurality of windows configured to expose the cavity to the environment, and thereby allow the fragrance strip to dispense its fragrance into the environment. The second section defines the inner perimeter and is configured with an inner surface that is shaped to complement the shape of a soap bottle. The cavity runs around the perimeter of the unitary structure and is bounded on one side (the outer side) by the plurality of windows, and on another side (the inner side) by the second section. A fragrance strip may be disposed within the cavity formed between the first and second sections, and exposed to the environment by the plurality of windows in the first section.

In a second illustrative embodiment of the present invention, a fragrance dispenser comprises a single section having an inner surface coupled to an outer surface to define a cavity, into which a fragrance strip may be disposed, and an opening through which a bottle may pass. In this second illustrative embodiment, the single section may be molded in a single piece, rather than from separate pieces joined together. The outer surface comprises a plurality of windows to expose the cavity to the environment, and a slit through which a fragrance strip may be inserted into the cavity. The inner surface may have a shape complementary to the shape of a bottle.

In the second illustrative embodiment, the section defines an opening with an outer perimeter having one or more teeth configured to engage with a threaded portion of a bottle. In this illustrative embodiment, the teeth are configured to engage with the threaded portion of the bottle, such that the fragrance dispenser removably couples to the bottle to form a unitary structure. This may allow the fragrance dispenser to be fixed to an existing fixture within a lavatory without the need for a separate mounting bracket.

This example is given to introduce the reader to the general subject matter discussed herein. The invention is not limited to this example. The following sections describe various embodiments of systems and methods for controlling aspects of a deformation of two-dimensional and three-dimensional images.

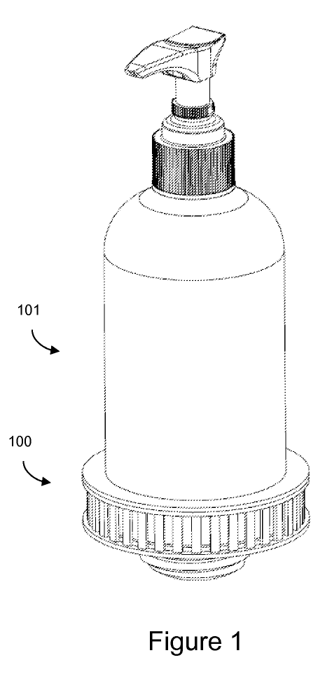

Referring now to the drawings in which like elements are labeled with like numerals throughout the several figures, FIG. 1 shows a soap bottle 101 engaged with a fragrance dispenser 100 according to one embodiment of the present invention. In the embodiment shown, fragrance dispenser 100 comprises features shown in detail in later figures, including an inner surface, and an outer surface coupled to the inner surface and defining a cavity between the two surfaces, the cavity configured to receive a fragrance strip. The outer surface also comprises at least one window configured to expose the cavity to the environment. In the embodiment shown, a fragrance strip is disposed within the cavity. Fragrance dispenser 100 allows a threaded portion of the bottle to extend through the fragrance dispenser and engage with a bottle base. In the embodiment shown, fragrance dispenser 100 is engaged to a thread on the base of the soap bottle 101 such that fragrance dispenser is secured to the base of the soap bottle.

In the embodiment shown in FIG. 1, fragrance dispenser 100 comprises polypropylene, however, other suitable materials, such as plastics or metals, may be used. For example, a fragrance dispenser according to one embodiment of the present invention comprises high-density polyethylene. Other embodiments may comprise other materials, such as plastics, composite materials, and/or metals.

Fragrance dispensers according to embodiments of the present invention may comprise one or more fragrance strips. Fragrance strips suitable for such fragrance dispensers may comprise a porous material, such as a paper or cotton-based material, in which an oil or other material may be impregnated to provide a fragrance. For example, in one embodiment of the present invention, a fragrance strip comprises 100 point paper and between 5 and 6 grams of an oil having a fragrance. In one embodiment, a fragrance strip may comprise a non-woven, porous material capable of being formed or cut into a shape suitable for insertion into the cavity. In one embodiment, a fragrance strip may comprise a solid gel formed or cut into a shape suitable for insertion into the cavity. A fragrance strip suitable for use within a fragrance dispenser according to embodiments of the present invention may have a shape complementary to a cavity formed within the fragrance dispenser. In one embodiment, a fragrance strip may occupy substantially all of the space within the cavity.

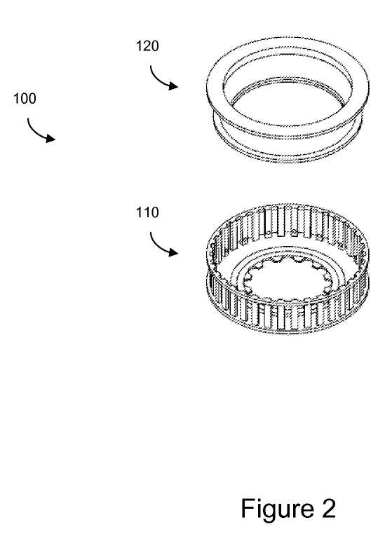

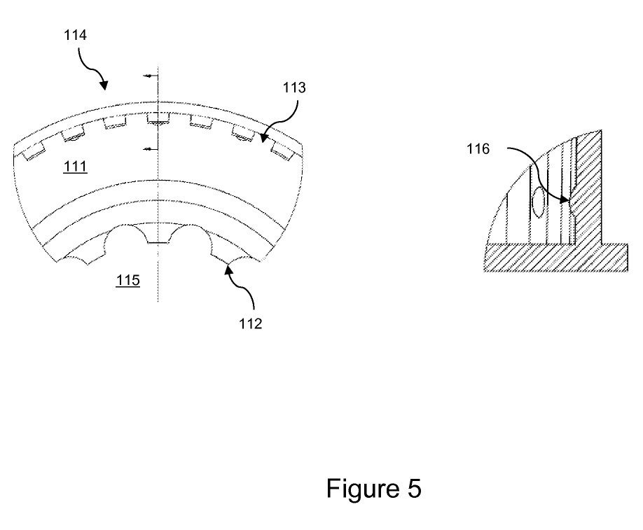

FIG. 2 shows a view of a fragrance dispenser 100 according to one embodiment of the present invention. In the embodiment shown, fragrance dispenser 100 comprises two sections, a first section 110 and a second section 120. Second section 120 is configured to be coupled to first section 110 to form a unitary structure. In the embodiment shown, second section 120 is configured to fit within first section 110 and to snap into place, as will be described in greater detail with respect to FIG. 5 below. In the embodiment shown, a cavity may be formed between the first and second sections such that a fragrance strip may be disposed within the cavity. The cavity and/or its contents may be exposed to the environment outside of the fragrance dispenser by one or more windows formed in the first section.

In the embodiment shown in FIG. 2, fragrance dispenser 100 comprises two sections, however, other embodiments may comprise only one section, or may comprise more than two sections. For example, in one embodiment of the present invention, fragrance dispenser comprises one section having an inner surface having a shape complementary to a bottle or other container, and an outer surface having one or more opening configured to expose a cavity defined between the inner and outer surfaces to the environment. In one embodiment of the present invention, fragrance dispenser comprises three sections. Still other embodiments having greater than or less than two sections, or sections having different configurations, are within the scope of the present invention.

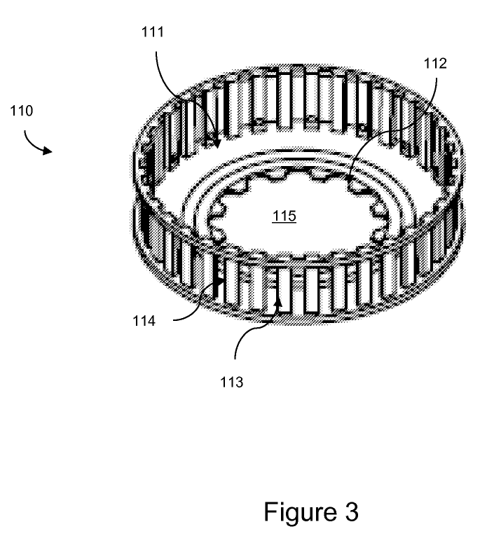

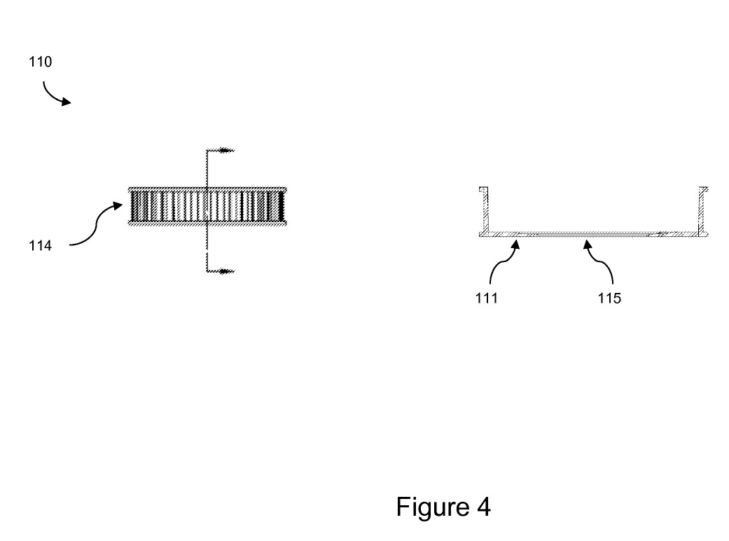

FIGS. 3 and 4 show detailed views of a first section 110 of a fragrance dispenser according to one embodiment of the present invention. The first section 110 shown in FIG. 3 comprises a base 111 and an outer surface 114 that defines a plurality of windows 1 13.

In the embodiment shown in FIG. 3, first section 110 defines an opening 115 in the base 111. The perimeter of the opening 115 comprises a plurality of teeth 112 configured to engage with a threaded portion of a bottle, such as the bottle 101 shown in FIG. 1. One embodiment of the present invention may define an opening having a perimeter having no teeth such that the perimeter is not configured to engage with a threaded portion of a bottle. In such an embodiment, a bottle having a threaded base may pass through the opening and engage with a base, into which the bottle may be screwed. The fragrance dispenser may then be held in place, such as by compression, between the bottle and the base. In another embodiment, the perimeter may not comprise any teeth, but may be configured to engage with a threaded portion of a bottle. For example, the diameter of the opening may be less than the diameter of a thread on a threaded portion of a bottle, but greater than the base of the bottle, which may allow the smooth perimeter of the fragrance dispenser to engage with the threaded portion of the bottle. Further embodiments configured to engage with the threaded portion of a bottle would be apparent to one of ordinary skill in the art.

FIG. 5 shows cross-sectional views of a first section 110 of a fragrance dispenser according to one embodiment of the present invention. In the embodiment shown, outer surface 113 comprises a feature 116 configured to engage with second portion 120 and to couple second section 120 to first section 1 10. The embodiment shown in FIG. 5 is configured to receive a second section 120 that comprises a protrusion configured to fit between feature 116 and the base 111 of the first section 1 10. In such a configuration, a protrusion or other portion of the second section 120 may slide over feature 116 and snap into place, such that feature 116 may help prevent second section 120 from unintentionally decoupling from first section 110. Other embodiments of the present invention may comprise different features for securing a second section to the first section. For example, in one embodiment, feature 116 may comprise a barbed shape, which may allow a second section to easily slide into place; however, the barb may substantially prevent a second section from being decoupled from a first section. In one embodiment according to the present invention, first and second sections may be coupled with an adhesive. In one embodiment, first and second sections may be welded together by heating the first and/or second sections such that a part of the heated section, or sections, melts and fuses with the opposing section.

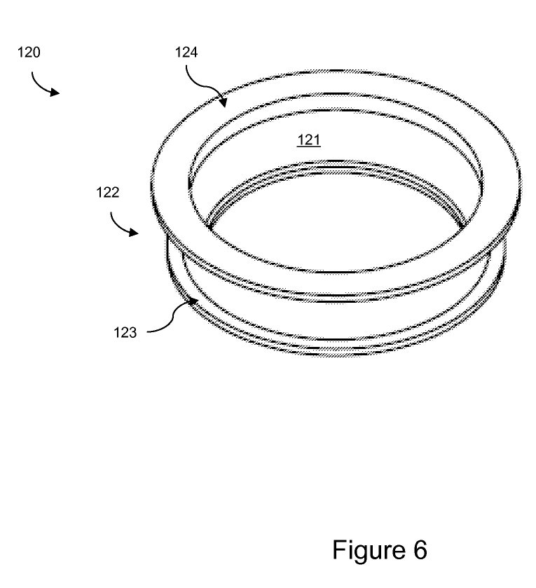

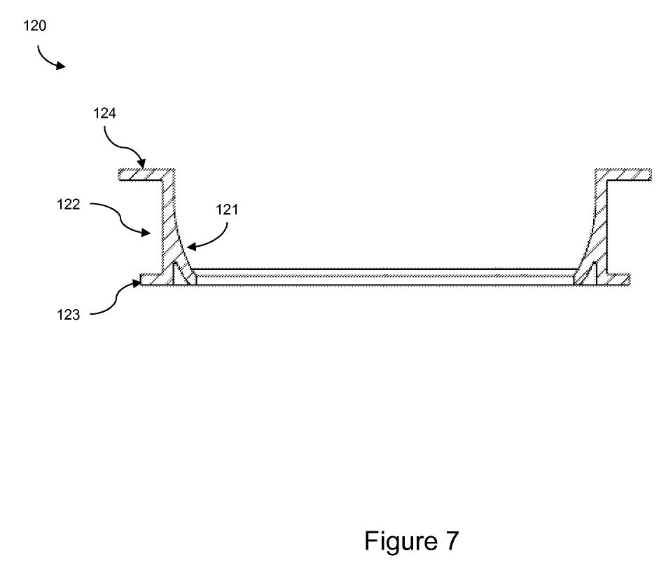

FIGS. 6 and 7 show a second section 120 of a fragrance dispenser according to one embodiment of the present invention. In the embodiment shown, second section 120 comprises a wall 122 having an inner surface 121, a lower surface 123, and an upper surface 124. In the embodiment shown, second section 120 comprises polypropylene, however, other suitable materials, such as plastics or metals, may be used. For example in one embodiment of the present invention, second section 120 comprises high-density polyethylene.

In the embodiment shown, and as can be seen more clearly in FIG. 7, inner surface 121 has a shape that may be complementary to the shape of a bottle. Second section 120 may be configured to help define a cavity when engaged with first section 1 10. For example, in the embodiment shown, lower surface 123 may engage with feature 116 of first section 110, while a cavity may be defined between wall 122 and outer surface 114 of first section 110. Upper surface 124 may engage with the upper surface of outer surface 114 such that the cavity formed may be closed. The defined cavity may be configured such that a fragrance strip may be disposed within the cavity, and exposed to the environment by windows 113 in first section 110.

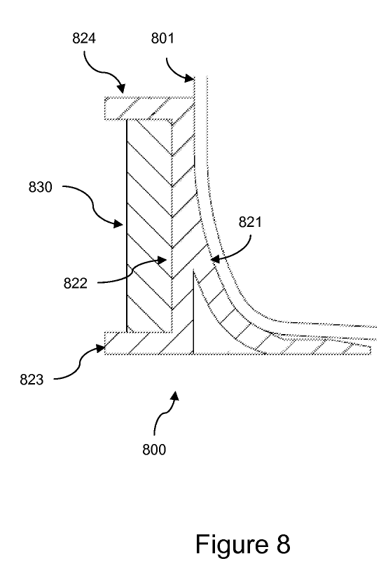

FIG. 8 shows a half cross-section of a fragrance dispenser 800 according to one embodiment of the present invention. In the embodiment shown, fragrance dispenser 800 comprises a substantially annular shape, and has a single section having an outer surface 822 and an inner surface 821. The fragrance dispenser also comprises an upper surface 824 and a lower surface 823. The perimeter of the fragrance dispenser 800 forms a trough between the upper and lower surfaces 824, 823 around which fragrance strip 830 may be wound. Inner surface 821 is configured to substantially complement the shape of a bottle 801, such as a soap bottle. In the embodiment shown, fragrance dispenser 800 defines an opening in the base through which a threaded portion of a bottle may pass. In one embodiment, fragrance dispenser 800 further defines one or more teeth (not shown) configured to engage with a threaded portion of the bottle. In one embodiment, fragrance dispenser 800 may be configured to engage with a base, rather than a bottle. In such an embodiment, inner surface 821 may be configured to substantially complement a shape of a base (not shown) to which a bottle may be coupled. For example, a base may be configured to receive and engage with a threaded portion of a bottle. Fragrance dispenser 800 may be configured to engage with such a base.

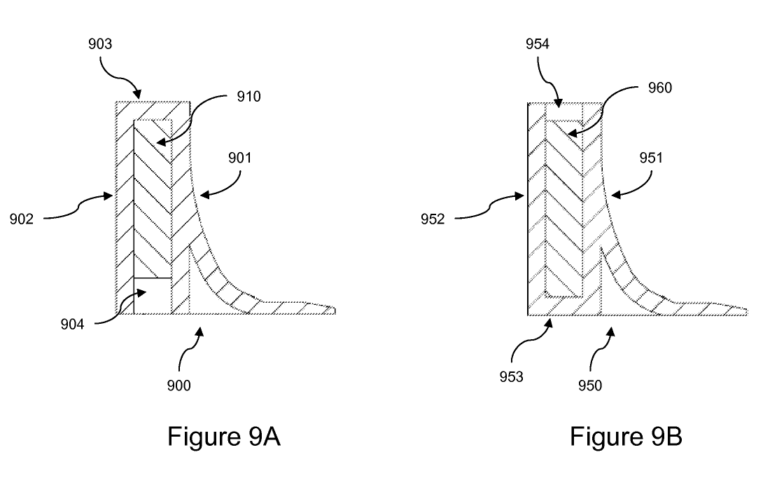

FIG. 9A shows a fragrance dispenser according to one embodiment of the present invention. In the embodiment shown, fragrance dispenser 900 has a substantially annular shape, and comprises an inner surface 901, an upper surface 903, and an outer surface 902, which define a cavity 904 between the inner and outer surfaces 901, 902. Outer surface comprises at least one window configured to expose the cavity 904 to the environment. Fragrance strip 910 may be disposed within the cavity 904. In the embodiment shown, cavity 904 is open at the base of the fragrance dispenser 900 and a fragrance strip 910 may be inserted into the cavity 904 through the opening. In the embodiment shown, the fragrance strip 910 may be held in place by compression between the inner and outer surfaces 901, 902. However, in other embodiments, the fragrance strip may be held in place by other means. For example, in one embodiment, fragrance dispenser 900 may define a cavity having a wide portion and a narrow portion. In such an embodiment, the narrow portion may define a ledge on which the fragrance strip 900 may rest. In one embodiment one or more feature or protrusions may be defined within cavity on either or both the inner and outer surfaces 901, 902. For example, in one embodiment, a plurality of barbs may be formed in the cavity on inner surface 901. The barbs may substantially support and hold in place the fragrance strip 910.

In the embodiment shown in FIG. 9A, fragrance dispenser 900 defines an opening configured to allow a threaded portion of a bottle to pass through. In such an embodiment, fragrance dispenser 900 may engage with a threaded portion of a bottle. In one embodiment, fragrance dispenser 900 may define one or more teeth configured to engage with a threaded portion of a bottle or of a base. In one embodiment, fragrance dispenser 900 may be configured to engage with a base, rather than a threaded portion of a bottle.

FIG. 9B shows a fragrance dispenser 950 according to one embodiment of the present invention. The embodiment is similar to the embodiment shown in FIG. 9A, however, the cavity 954 defined in fragrance dispenser 950 is open at the top, rather than at the bottom, and may receive fragrance strip 960 through the opening. In the embodiment shown, cavity 954 is defined by inner surface 951, outer surface 952, and lower surface 953. Lower surface 953 may further support and hold in place fragrance strip 960. Similar to fragrance dispenser 900, fragrance dispenser 950 may define an opening to allow a threaded portion of a bottle to pass through and engage with a base. In one embodiment, fragrance dispenser 950 may define one or more teeth in the opening configured to engage with a threaded portion of a bottle. In one embodiment, fragrance dispenser 950 may be configured to engage with a base.

Fragrance dispenser 950 may have the advantage that insertion of a fragrance strip may be easier than fragrance dispenser 900. Further, fragrance dispenser 960 may further have the advantage that additional features, protrusions, or ledges may not be necessary to secure fragrance strip 960 within cavity 954. However, such embodiments may be more prone to accumulate debris within the cavity 954 or to tampering with or removal of the fragrance strip 960. Embodiments of the present invention similar to fragrance dispenser 900 may have an advantage in that fragrance dispenser 900 may be more resistant to tampering as the fragrance strip 910 is not easily accessible, or may not be obviously accessible to a user of a bottle, such as a soap dispenser. However, it may be more difficult to insert a fragrance strip 910 within a cavity 904 of such embodiments as it may require decoupling the fragrance dispenser 900 from a bottle or base, and inverting the fragrance dispenser 900 to insert the fragrance strip 910.

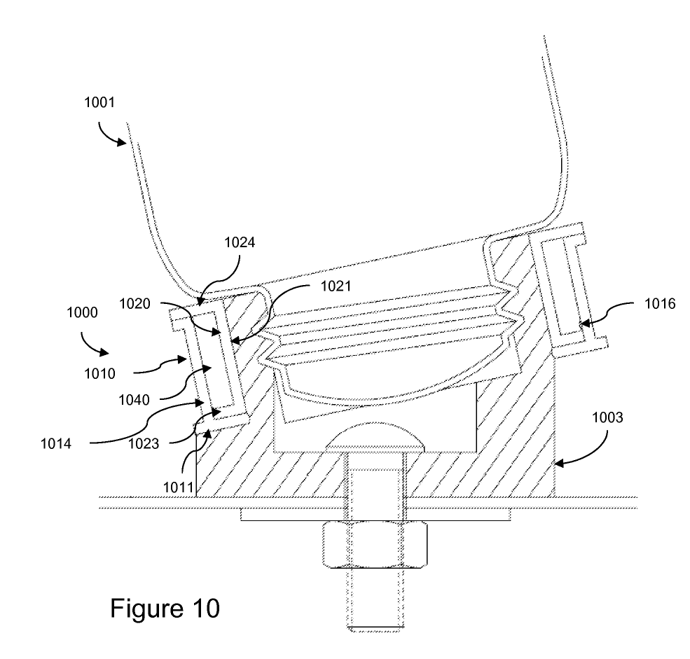

Referring now to FIG. 10, which shows a cross section of a fragrance dispenser 1000 coupled to a base according to one embodiment of the present invention. In the embodiment shown, fragrance dispenser 1000 has a substantially annular shape, and comprises a first section 1010 and a second section 1020. In the embodiment shown in FIG. 10, second section 1020 is configured to be coupled to first section 1010 to form a unitary structure and to define a cavity 1040. First section 1010 comprises an outer surface 1014 and a base 1011. In the embodiment shown, outer surface 1014 defines at least one window configured to expose cavity 1040 to the environment. Outer surface 1014 further comprises at least one feature 1016 configured to engage with second section 1020 to couple second section 1020 to first section 101 0. In the embodiment shown, feature 1016 comprises a protrusion from the interior surface of outer surface 1010. In one embodiment feature 1016 comprises a different shape. For example, in one embodiment, feature 1016 comprises a barbed shape such that second section 1020 may easily slide past feature 1016, but may be secured in place by the underside of the barbed shaped feature. In one embodiment of the present invention, outer surface 1010 does not comprise any features and second section 1020 may be coupled to first section 1010 by frictional forces, or may be held in place by gravity.

Base 1011 of first section 1010 may define an opening configured to allow a base 1003 and a threaded portion of a bottle 1001 to pass through the opening. For example, in the embodiment shown in FIG. 10, base 1011 defines a substantially circular opening configured to receive base 1003 and threaded portion of bottle 1001, such that bottle 101 may screw into and couple to base 1003.

In the embodiment shown in FIG. 10, second section 1020 comprises an inner surface 1021, an upper surface 1024, and a lower surface 1023. Like first section 1010, second section 1020 comprises a substantially annular shape, and defines an opening through which a base 1003 and a threaded portion of a bottle 1001 may pass.

In the embodiments shown, fragrance dispensers 100, 1000 have substantially annular shapes; however, other embodiments may comprise different shapes. For example, in one embodiment of the present invention, a fragrance dispenser may comprise a square or rectangular shape. In one embodiment, a fragrance dispenser may comprise a triangular or other polygonal shape. In such embodiments, the fragrance dispenser may define an opening to receive a threaded portion of a bottle, such as a soap dispenser. In the embodiments described herein, the opening has been substantially circular; however, fragrance dispensers according to other embodiments may comprise differently-shaped openings. For example, in one embodiment, a fragrance dispenser may define a square or rectangular opening. Such an embodiment may be advantageous when coupling the fragrance dispenser to a base having a complementary shape. Still other shaped openings may be defined as well including triangular, polygonal, or other curved shapes, such as a “figure 8” outline. Further, a shape of a fragrance dispenser need not have the same shape as the opening defined by the fragrance dispenser. For example, in one embodiment, a fragrance dispenser may have a substantially square shape, but define a substantially triangular opening. In one embodiment a fragrance dispenser may comprise an annular shape, but define a square opening. Still further embodiments would be apparent to one of ordinary skill in the art.

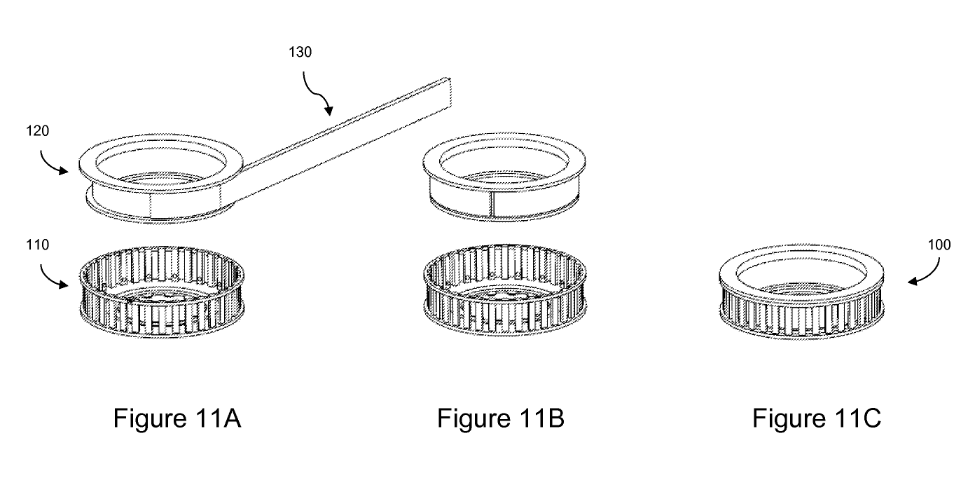

FIGS. 11A-C show a method of assembly of a fragrance dispenser according to one embodiment of the present invention. In the embodiment shown in FIGS. 11A-C, a fragrance dispenser comprises a first section 110, a second section 120, and a fragrance strip 130. In FIG. 11A, fragrance strip 130 is engaged with wall 122 of second section 120. In FIG. 11B, fragrance strip 130 is fully engaged with and wrapped around second section 120. In FIG. 11C, second section 120, with fragrance strip 130, is engaged with first section 110 such that fragrance strip is disposed within a cavity defined between first section 110 and second section 120, and is exposed to the environment external to the cavity by a plurality of windows 113. In the embodiment shown, lower surface 123 of second section 120 is engaged with feature 115 of first section 110.

In one embodiment of the present invention, a fragrance dispenser may have an outer surface comprising a plurality of windows and a slit. In such an embodiment, a fragrance strip may be inserted into a cavity formed within the fragrance dispenser through the slit, such that the fragrance strip may be inserted into the fragrance dispenser after the two sections have been coupled.

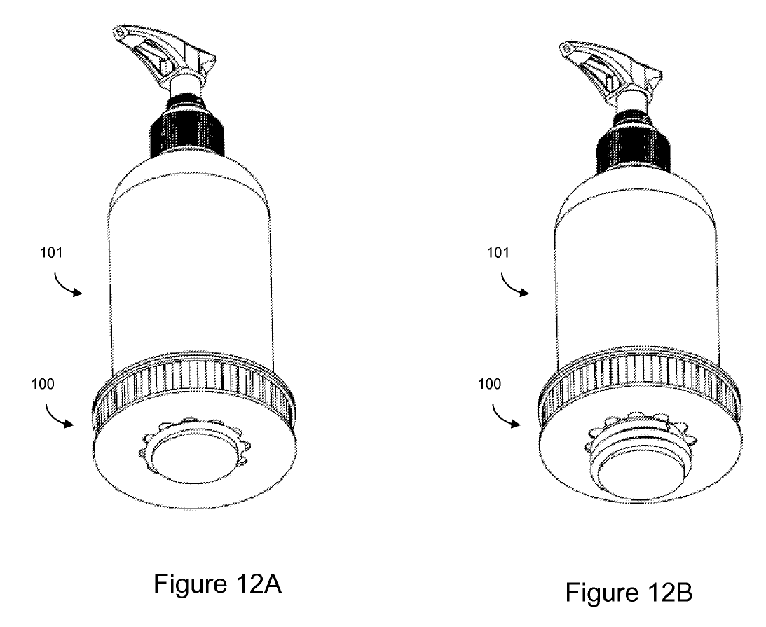

FIGS. 12A-B show a method of engaging a fragrance dispenser to a threaded portion of a bottle according to one embodiment of the present invention. In the embodiment shown, fragrance dispenser 100 engages with a threaded portion of bottle 101. Fragrance dispenser 100 may then be screwed onto the threaded portion of the bottle 101. In such a configuration, fragrance dispenser may remain secured to the bottle 101 and may be resistant to decoupling. A fragrance dispenser according to one embodiment of the present invention may not engage with a threaded portion of a bottle. In such an embodiment, the fragrance dispenser may be configured to be held in place between a bottle and a base by compression. In one embodiment of the present invention, a fragrance dispenser may define an opening having a diameter greater than a diameter of a base, but less than a diameter of a bottle, such that the fragrance dispenser is configured to be placed over the base, but will not pass over the bottle, due to the size of the bottle.

Embodiments of the present invention may provide advantages over existing fragrance dispensers. For example, in one embodiment, a fragrance dispenser may couple to a bottle, such as a soap dispenser in an airplane's lavatory, by engaging with a threaded portion of the bottle. Additionally, a fragrance dispenser according to embodiments of the present invention may not require a separate mounting bracket, but instead may engage with an existing fixture, such as coupling to a bottle screwed into a base. A further advantage may be that the fragrance strip provides more efficient dispersal of fragrance into the environment because of being disposed in cavity around the perimeter of the dispenser, and being exposed to the environment through a plurality of windows. Additionally, because the fragrance dispenser, in some embodiments, engages with a threaded portion of a bottle, a fragrance dispenser may have a low profile relative to the height of the bottle so as to not interfere with labeling on the bottle, including branding information, instructions, or warning information.

The foregoing description of the embodiments, including preferred embodiments, of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the invention.

That which is claimed is:

1. A fragrance dispenser, comprising:
an inner surface; and
an outer surface defining a plurality of windows, the outer surface coupled to the inner surface such that a cavity is defined between the inner surface and the outer surface, the cavity configured to receive a fragrance strip,
wherein the fragrance dispenser is configured to allow a threaded portion of a bottle to extend through the fragrance dispenser and engage with a bottle base.

2. The fragrance dispenser of claim 1, further comprising a first section and a second section, the first section separate from the second section, wherein the first section comprises the outer surface, and the second section comprises the inner surface.

3. The fragrance dispenser of claim 2, wherein the first section comprises a base, which defines an opening having a perimeter, the opening configured to allow the a threaded portion of a bottle to extend through the fragrance dispenser and engage with a bottle base

4. The fragrance dispenser of claim 3, wherein the perimeter of the opening comprises a plurality of teeth configured to engage with the threaded portion of the bottle.

5. The fragrance dispenser of claim 3, wherein the opening is configured to not engage with the threaded portion of the bottle.

6. The fragrance dispenser of claim 1, further comprising a fragrance strip disposed within the cavity.

7. The fragrance dispenser of claim 6, wherein the fragrance strip comprises paper and a fragrance.

8. The fragrance dispenser of claim 1, further comprising a bottle having a threaded portion.
